(19) United States
(12) Patent Application Publication
(10) Pub. No.: US 2013/0108628 A1
(43) Pub. Date: May 2, 2013

(54) Method for treating cancer pain and/or rescuing analgesic effect of morphine treatment of cancer pain

(71) Applicant: Xue-Jun Song, Irving, TX (US)

(72) Inventor: Xue-Jun Song, Irving, TX (US)

(21) Appl. No.: 13/373,018

(22) Filed: Nov. 2, 2011

(51) Int. Cl. A61K 39/395; A61P 25/04

(52) U.S. Cl. 424/134.1

(57) ABSTRACT

The present invention provides a method for treating cancer pain by administering to an individual in need of such treatment with a pharmaceutically effective amount of a blocking reagent for ephrinB-EphB signaling. Also provided is a method for reducing opioid tolerance in treating cancer pain thereby rescuing analgesic effect of morphine treatment of cancer pain by administering to an individual in need of such treatment with a pharmaceutically effective amount of a blocking reagent for ephrinB-EphB signaling. The blocking reagent can be an EphB receptor blocker such as EphB1-Fc and EphB2-Fc.

### BACKGROUND OF THE INVENTION

1. Field of the Invention

The present invention generally relates to medical treatment. Particularly, the present invention relates to a method for treating cancer pain and/or rescuing analgesic effect of morphine treatment of cancer pain using a blocking reagent for ephrinB-EphB signaling.

2. Description of the Related Art

Tumor cells act to cause pain in many ways. For example, bone cancer pain is one of the most common symptoms presented by patients with primary bone sarcomas or secondary bone cancers that predominantly occur as distant metastases of non-bone primary tumors, notably those in breast, prostate and lung. Production of prostaglandin and other molecules by tumors, tumor-associated macrophages and other host cells stimulate osteoclast-mediated bone resorption. Nociceptors in bone are stimulated via activation of transient receptors potential vanilloid type-1, endothelin A and TrkA receptor. Activation is directed by acid microenvironment, endothelin-1, and nerve growth factor, respectively. Mechanisms of bone cancer pain are complex and may involve a combination of inflammatory and neuropathic pain with unique characteristics. For instance, bone cancer pain markedly increases expression of glial fibrillary acidic protein (GFAP), but not substance P and calcitonin gene-regulated peptide (CGRP) in the dorsal horn (DH) of the spinal cord or galanin and neuropeptides Y in dorsal root ganglion (DRG) neurons. Despite decades of thorough study, the specific cellular and molecular mechanisms underlying bone cancer pain remain elusive and the clinical approaches for treating bone cancer pain are limited.

Eph receptors, named for its expression in an erythropoietin-producing human hepatocellular carcinoma cell line, consist of the largest family of receptor tyrosine kinases, which play vital roles in transmitting external signals to the interior of many types of cells. EphB receptors and their ligands ephrinBs are critical cues in regulating cell shape, adhesion/repulsion, migration and positioning during developmental processes. Alteration of mechanisms controlling adhesion and motility has a central role in promoting tumor invasion and angiogenesis. Altered expression of ephrins and/or Eph receptors has been implicated in tumor progression in a number of human malignancies. Recently, ephrinB-EphB receptor signaling has been discovered to be involved in pain after nerve injury or opiate withdrawal. EphrinB-EphB interaction is well known to lead bidirectional signals, the forward- and the reverse-signaling. However, there is no clear evidence if the forward- or the reverse-signaling or both is necessary for production and/or persistence of any pain status.

Opioid drugs are used and abused for their analgesic and rewarding properties. Repeated use of opioids such as morphine for relief of chronic pain can lead to opiate tolerance and dependence. Despite decades of investigation, the specific cellular and molecular mechanisms underlying opioid tolerance and withdrawal-induced pain enhancement remain elusive. It is of clinical important to determine the mechanisms underlying tolerance and physical dependence, and to develop medications that can prevent, minimize, or reverse opioid tolerance and dependence.

There is apparently a need for an effective method for treating cancer pain and/or for reducing opioid tolerance in treating cancer pain thereby rescuing analgesic effect of morphine treatment of cancer pain. The present invention fulfills this long-standing need.

### SUMMARY OF THE INVENTION

The present invention is directed to a method for treating cancer pain by administering to an individual in need of such treatment with a pharmaceutically effective amount of a blocking reagent for ephrinB-EphB signaling.

The present invention is also directed to a method for reducing opioid tolerance in treating cancer pain thereby rescuing analgesic effect of morphine treatment of cancer pain by administering to an individual in need of such treatment with a pharmaceutically effective amount of a blocking reagent for ephrinB-EphB signaling.

The blocking reagent can be an EphB receptor blocker such as EphB1-Fc and EphB2-Fc.

The foregoing and other advantages of the present invention will be apparent to those skilled in the art, in view of the following detailed description of the preferred embodiment of the present invention, taken in conjunction with the accompanying drawings.

### BRIEF DESCRIPTION OF THE DRAWINGS

The present application file contains at least one drawing executed in color. Copies of this patent application publication with color drawing(s) will be provided by the Office upon request and payment of the necessary fee.

Features of the present invention as well as a preferred mode of use, further objectives, and advantages thereof, will best be understood by reference to the following detailed description of an illustrative embodiment when read in conjunction with the accompanying drawing, wherein:

FIGS. 1A-1D illustrate upregulation of EphB1 and ephrinB2 in the spinal cord following tumor cell implantation (TCI). FIGS. A-B: Examples of Western blot analysis and data summary show protein expression and phosphorylation of EphB1 (A) and ephrinB2 and PY99 (B) in a time-dependent manner following TCI. FIGS. C-D: Confocal images of immunostaining for EphB1 (green) and ephrinB2 (green) and their colocalization with neurons (NeuN, red) ipsilateral to TCI. Magnification: 100×(C); 200×(D).

FIGS. 2A-2D illustrate distribution and colocalization of expression of EphB1 and ephrinB2 with cells and primary afferents within the DH and DRG after TCI. FIG. 2A: EphB1 (green) with astrocytes (GFAP, red, left) and, in a small amount, with microglial cell (IBA, red, right). FIG. 2B: EphrinB2 (green) with astrocytes (red, left), but not microglial cell (red, right). FIGS. 2C-2D: EphrinB2 (green) with primary afferents CGRP (red), but not IB4 (red) in the DH (C) and with CORP (red)-, but not IB4 (red)-nociceptive cells in the DRG (D). Magnification: 200×.

FIGS. 3A-3D illustrate that spinal blocking EphB receptor activation prevents and attenuates pain-related behaviors after TCI. Thermal hyperalgesia (FIGS. 3A-3B) and mechanical allodynia (FIGS. 3C-3D) are shown in the feet ipsilateral to TCI. Administration of EphB2-Fc or its Fc control (each 5 μg, i.t.) on the postoperative 3, 4 and 5 days (FIGS. 3A and 3C) or 7, 8 and 9 days (FIGS. 3B and 3D) are indicated by the arrows. TCI was performed on day 0 (▴). Eight rats were included in each group. *P<0.05, **P<0.01 indicate significant differences compared to the group of control (Sham+Fc). #P<0.05, ##P<0.01 indicate significant differences compared to the corresponding TCI group.

FIGS. 4A-4D illustrate that blocking EphB receptor activation prevents and attenuates pain-related behaviors after TCI. FIGS. 4A-4B: spontaneous guarding and flinching. FIGS. 4C-4D: movement-evoked guarding and limb use. Data shows the foot ipsilateral to TCI tested on the postoperative 14 day. Eight rats were included in each group. *P<0.05, **P<0.01 indicate significant differences compared to the control group (Sham+Fc). #P<0.05, ##P<0.01 indicate significant differences compared to the corresponding TCI group.

FIGS. 5A-5C illustrate that blocking EphB receptor activation suppresses induction of c-Fos and activation of astrocytes and microglial cells in the DH ipsilateral to TCI. FIG. 5A: induction of c-Fos. FIG. 5B: activation of astrocytes (GFAP). FIG. 5C: activation of microglial (IBA). EphB2-Fc (5 μg, i.t., once a day) were given on postoperative 7, 8 and 9 days, respectively. Tissues were collected on the 14th day after TCI. Magnifications: 200×. **P<0.01 indicates significant differences compared to the corresponding control of Sham+Fc. ##P<0.01 indicates significant differences compared to the corresponding control of TCI+Fc.

FIGS. 6A-6B illustrate presence of osteolytic lesions in the tibia of the rats after TCI. FIG. 6A: X-ray images show bone destruction and tumor growth on postoperative 14 and 21 days. FIG. 6B: HE staining shows bone destruction and tumor growth on the 14th day after TCI. Scale bar in A: 100 μm. EphB2-Fc (5 μg, i.t., once a day) was given on postoperative 7, 8 and 9 days, respectively.

FIGS. 7A-7B illustrate that blocking EphB receptor activation suppresses TCI-induced upregulation of pNR1, pNR2B, pSrc(Tyr418), pERK1/2, pCaMKII, and pCREB in the spinal cord. EphB2-Fc (5 μg, i.t., daily) was administrated at postoperative 7, 8 and 9 day, respectively. Four samples were included in each of pNR1, pNR2B, and pSrc(Tyr418) groups and three in the other groups. *P<0.05, **P<0.01 indicate significant differences compared to the control (Sham and Sham+Fc). #P<0.05, ##P<0.01 indicate significant differences compared to the corresponding group of TCI+Fc on the specific day indicated.

FIGS. 8A-8C illustrate that spinal administration of EphB2-Fc inhibits TCI-induced upregulation of expression of EphB1, but not ephrinB2. FIG. 8A: a single dose of EphB2-Fc or its Fc control (each 5 μg) was injected on the 7th day after TCI. FIGS. 8B-8C: repetitive EphB2-Fc (EphB2-Fcx3, each 5 μg, once a day) were injected on postoperative 7, 8, and 9 day, respectively. Tissues were collected 2 h after the last treatment with EphB2-Fc or Fc. **P<0.01 indicates significant differences compared to the corresponding group of Sham+Fc. #P<0.05, ##P<0.01 indicate significant differences compared to the corresponding group of TCI+Fc.

FIGS. 9A-9D illustrate that EphB1 receptor activation is required for ephrinB2-Fc in activating NR1 and NR2B receptors in naïve rat spinal cord. FIGS. 9A-9B: spinal administration of ephrinB2-Fc (2 μg) activated EphB1 (A), NR1 and NR2B (B). FIGS. 9C-9D: pretreatment with EphB2-Fc (5 μg, i.t., daily for 3 consecutive days) blocked ephrinB2-Fc-induced activation of EphB1 (C), NR1 and NR2B (D). Four samples were included in each of the groups. Tissues were taken 4 h after the last dose of EphB2-Fc and ephrinB2-Fc, respectively, in C and D. **P<0.01 indicates significant differences compared to the corresponding control group (both Nave and Fc).

FIGS. 10A-10B illustrate that spinal administration of ephrinB2-Fc down-regulates ephrinB2 in the spinal cord and induces thermal hypersensitivity in naïve rats. FIG. 10A: Western blot analysis shows expression of ephrinB2 and its phosphorylation, PY99. EphrinB2-Fc: 2 μg, i.t. Tissues were taken at 1 h, 2 h and 4 h after injection. Four samples were included in each group. FIG. 10B: single dose of ephrinB2-Fc (2 μg) caused thermal hypersensitivity. Eight rats were included in each group. **P<0.01 indicate significant differences compared to the corresponding control groups.

FIGS. 11A-11D illustrate that repetitive spinal administration of ephrinB2-Fc increases level of EphB1, pNR1 and pNR2B and decreases ephrinB2 in the spinal cord while causing thermal hypersensitivity in naïve rats. FIGS. 11A-11C: Western blot shows effects of ephrinB2-Fc on expression of EphB1 and ephrinB2 and their phosphorylation and phosphorylation of NR1 and NR2B. Tissues were taken 4 h after the third dose of ephrinB2-Fc. **P<0.01 indicates significant differences compared to the corresponding control groups of PBS and Fc.

FIGS. 12A-12B illustrate that activation of EphB1 receptor is required for ephrinB2-Fc inducing thermal hypersensitivity. Pretreatment with EphB2-Fc (each 5 μg, i.t., once a day for 3 consecutive days) blocked ephrinB2-Fc (2 μg)-induced thermal hypersensitivity (A), but not the accompanied downregulation of ephrinB2 (B). Eight rats were included in each group and injection(s) was indicated by the arrow(s) in A. Four samples were included in each group in and tissues were taken 4 h after injection of ephrinB2-Fc in B. **P<0.01 indicates significant differences compared to any of the control groups.

FIGS. 13A-13D illustrate effects of MMP-2 and MMP-9 on expression of EphB1. FIGS. 13A-13B: MMP2i and FN439 reversed EphB2-Fc-induced downregulation of EphB1 in naïve (A) and TCI rats (B). FIG. 13C: effects of MMP2i and FN439 on EphB1 in lack of EphB2-Fc treatment in naïve (left) and TCI (right) rats. FIG. 13D: effects of MMP-2 and MMP-9 on EphB1 without EphB2-Fc treatment in naïve (left) and TCI (right) rats. Four samples were included in each group. Tissues were taken 4 h after the last treatment. *P<0.05, **P<0.01 indicate significant differences compared to the corresponding control group (Naïve or Sham). #P<0.05, ##P<0.01 indicate significant differences compared to the corresponding control group (PBS in A and TCI in B).

FIGS. 14A-14B illustrate Spinal administration of EphB2-Fc or ephrinB2-Fc upregulates activity of MMP-2 and MMP-9. G elatin zymography shows activity of MMP-2 and MMP-9 in the spinal cord in naïve rats. Intensity of both MMP-9 and active MMP-9 as well as MMP-2 and active MMP-2 are measured and included in A and B. Tissues were taken 4 h after spinal injection of EphB2-Fc (5 μg) or ephrinB2-Fc (2 μg). Four samples were included in each group. *P<0.05, **P<0.01 indicates significant differences compared to the corresponding groups of PBS and Fc.

FIGS. 15A-15D illustrate that spinal administration of EphB2-Fc rescues analgesic effect of morphine (Mor) in treating TCI-induced thermal hyperalgesia in rats and mice' defensive pain. FIG. 15A: effects of EphB2-Fc and Mor on thermal hyperalgesia. Mor: 10 mg/kg, s.c., twice a day. EphB2-Fc: 2 μg, t., once a day. Eight rats were included in each group. FIG. 15B: effects of EphB2-Fc and Mor on expression of EphB1. Five samples were included in each group. FIGS. 15C-15D: effects of EphB2-Fc or targeted mutation of EphB1 on acute (Mor100-10) (C) and chronic (Mor10) (D) Mor tolerance. Data were calculated as percentage of maximal possible effect [MPE (%)]. Ten mice were included in each group. EphB2-Fc or its Fc control (each 2 μg) was administrated 30 min before the first dose of Mor in acute tolerance or each of Mor injection in chronic tolerance model. *p<0.05, **p<0.01 indicate significant differences compared to the corresponding control group, PBS (A), Naïve (B), Mor or EphB1^{+/+} (C-D). #p<0.05, ##p<0.01 indicate significant differences compared to the corresponding group of Mor.

FIGS. 16A-16C illustrate spinal administration of EphB2-Fc inhibits morphine (Mor)-induced upregulation of EphB1 in rats. FIG. 16A: Mor upregulates expression of EphB1 and its phosphorylation. Mor100: a single treatment of Mor (100 mg/kg, s.c.). Mor(100-10): Mor at 10 mg/kg was injected 24 h after the first dose at 100 mg/kg (acute tolerance protocol). Tissues were taken 0.5 h after the second injection. FIG. 16B: EphB2-Fc inhibits Mor-induced upregulation of EphB1. EphB2-Fc (2 μg and 5 μg, i.t.) was administrated 30 min before the first dose of Mor at 100 mg/kg. FIG. 16C: EphB2-Fc (5 μg, i.t.) was administrated 30 min before each of Mor at 10 mg/kg. Mor10: chronic tolerance protocol. Five samples were included in each group. *p<0.05, **p<0.01 indicate significant differences compared to group of Sham. #p<0.05, ##p<0.01 indicate significant differences compared to the corresponding group of Fc (B, C).

### DETAILED DESCRIPTION

The present invention demonstrates that EphB1 receptor forward signaling in the spinal cord is critical to the development of cancer pain (e.g. bone cancer pain) and morphine tolerance in treating the cancer pain. Tibia bone cavity tumor cell implantation (TCI) produces bone cancer-related thermal hyperalgesia, mechanical allodynia, spontaneous and movement-evoked pain behaviors as well as bone destruction. Production and persistence of these pain behaviors are well correlated with TCI-induced upregulation of EphB1 receptor and its ligand ephrinB2 in the dorsal horn and primary sensory neurons. Spinal administration of an EphB1 receptor blocking reagent (e.g. EphB2-Fc) prevents and reverses bone cancer-pain behaviors and the associated induction of c-Fos and activation of astrocytes and microglial cells, NR1 and NR2B receptors, Src within the NMDAR complex, and the subsequent Ca²⁺-dependent signals. The exogenous ligand ephrinB2-Fc upregulates level of phosphorylation of NR1 and NR2B receptors depending on the activation of EphB1 receptor. Spinal administration of EphB2-Fc and ephrinB2-Fc induces downregulation of EphB1 and ephrinB2, respectively, accompanied with increased activity of matrix metalloproteinase (MMP)-2/9. Blocking MMP-2 or MMP-9 reverses EphB1-Fc treatment-induced downregulation of EphB1 receptor. In addition, spinal blocking or targeted mutation of EphB1 receptor reverses morphine tolerance in treating bone cancer pain in rats and, in mice, defensive pain. These findings demonstrate a critical mechanism underlying the pathogenesis of bone cancer pain and suggest a potential target for treating cancer pain (e.g. bone cancer pain) and improving analgesic effect of morphine clinically.

The present invention is directed to a method for treating cancer pain by administering to an individual in need of such treatment with a pharmaceutically effective amount of a blocking reagent for ephrinB-EphB signaling.

The present invention is also directed to a method for reducing opioid tolerance in treating cancer pain thereby rescuing analgesic effect of morphine treatment of cancer pain by administering to an individual in need of such treatment with a pharmaceutically effective amount of a blocking reagent for ephrinB-EphB signaling.

The blocking reagent can be an EphB receptor blocker, two examples of which are EphB1-Fc and EphB2-Fc. The blocking reagent can be administrated intrathecally to the individual in need of such treatment in the dosage range of from about 0.5 μg to about 75 mg, preferably, from about 1 μg to about 15 mg. The individual being treated is usually a mammal, for instance, a human.

Examples of cancers include primary bone sarcomas and secondary bone cancers that predominantly occur as distant metastases of non-bone primary tumors, notably those in breast (breast cancer), prostate (prostate cancer) or lung (lung cancer) of the individual in need of the treatment.

The following example is given for the purpose of illustrating various embodiments of the invention and is not meant to limit the present invention in any fashion.

### EXAMPLES

### Materials and Methods

### Animals

All animals were used in accordance with the regulations of the ethics committee of the International Association for the Study of Pain and all protocols approved by the Institutional Animal Care and Use Committees. Adult, male, Sprague-Dawley rats, adult, male and female CD-1 mice (Charles River Laboratories) and the EphB1^{-/-} and EphB1^{+/+} mice (Henkemeyer Laboratory at University of Texas Southwestern Medical center) were used in this study. All surgeries were done under anesthesia with pentobarbital (Sigma, 50 mg/kg, i.p.).

### Therapeutic Reagents

An EphB1 receptor blocking reagent EphB2-Fc and an EphB1 receptor activator ephrinB2-Fc were used to determine the possible roles of EphB1 receptor in TCI-induced pain-like behaviors and the associated neurochemical alterations. EphB2-Fc possibly combines with the endogenous ephrinBs and thus EphB1 is substituted and cleaved. This may result in inhibition of the downstream signals of EphB1. EphrinB2-Fc possibly combines with EphB1 receptor to activate EphB1 receptor and the downstream signals. Inhibitors for MMP-2 (MMP2i) and MMP-9 (FN439) were used to determine possible roles of MMP-2/9 in modulation of EphB1. Each of the drugs used was dissolved in PBS or DMSO and then injected intrathecally (i.t. 10 μl for mice and 20 μl for rats) by means of lumbar puncture at the intervertebral space of L₄₋₅ and L₅₋₆ for multiple injections. These drugs and their final concentrations were: EphB2-Fc (2 or 5 μg), ephrinB2-Fc (2 μg), FN439 (100 μg), MMP2i (10 μg), DMSO (1%) (Sigma-Aldrich) and IgG-Fc (2 or 5 μg, Jackson laboratories).

### Model of Bone Cancer Pain

Tumor cells were extracted from ascitic fluid of rats that received Walker-256 rat mammary gland carcinoma cells. Tumor cells implantation (TCI) was mimicked by injecting the cells (1×10⁵ cells/μl, 5 μl) into the intra-medullary space of the right tibia to induce bone cancer in rats (Medhurst S J, et al., Pain 2002; 96:129-40).

### Assessment of Bone Cancer-Related Pain Behaviors

Thermal hyperalgesia was determined by the significant shortened latency of foot withdrawal in response to heat stimulation. Mechanical allodynia was indicated by a significant decrease in the threshold of paw withdrawal to mechanical indentation of the plantar surface of each hindpaw. The protocols were similar to those described previously (Song X J, et al., J Neurophysiol 2006; 95:479-92; Wang Z B, et al., Pain 2005; 114:266-77).

### Spontaneous and Movement-Evoked Pain-Like Behaviors

Spontaneous and movement-evoked pain-like behaviors were analyzed as described previously (Kato A, et al., Oncology 2008; 74 Suppl 1:55-60). Spontaneous nocifensive behaviors were evaluated by measuring spontaneous guarding and flinching over a 2-min period of observations. Movement-evoked pain was assessed by measuring the time spent guarding over a 2-min period of observations after non-noxious palpation, and the limb use during spontaneous ambulation, which was scored on a scale of 0-4: 0=normal use, 1=slightly limping, 2=clearly limping, 3=no use of the limbs (partial), and 4=no use of the limbs.

### Western Blotting

Protein precipitation procedures in conjunction with Western blots were employed to identify temporal expression of EphB1, ephrinB2 protein and their phosphorylation. Protocols were similar to those described previously (Liu W T, et al., FASEB J 2009; 23:90-8; Liu W T, et al., J Neurosci 2010; 30:7613-23). EphB1 and ephrinB2 were immunoprecipitated from 2 mg total protein/int tissue lysate using an anti-EphB1 antibody (2 μg; Santa Cruz Technology, SCT) complex with protine G-agarose (Invitrogen) and wheat germ agglutinin (WGA) linked to agarose (Sigma), respectively. EphrinB2 and its phosphorylation were detected using anti-ephrinB2 (C20, SCT) and anti-phosphotyrosine antibody PY99 (SCT), respectively (Georgakopoulos A, et al., EMBO J 2006; 25:1242-52). EphB1 and its phosphorylation were detected using anti-EphB1 antibody (Q20, SCT) and phosphotyrosine antibody 4G 10 (Upstate Biotechnology), respectively (Compagni A, et al., Dev Cell 2003; 5:217-30). NR2A immunoprecipitation was performed using anti-NR2A antibody followed by adsorption to protein G-agarose. Whole cell protein extracts lysates were used to identify temporal expression of the phosphorylated protein levels of NR1(pNR1), NR2B(pNR2B), Src(pSrc), ERK(pERK), CaMKII(pCaMKII), CREB(pCREB) and GAPDH. The primary antibodies were used include pNR1(Ser897), 1:800 (Millipore); pNR2B(Tyr1472), 1:300 (Chemicon); pSrc(Tyr418), 1:800 (Abeam, Cambridge, UK); pERK1/2(Thr202/Tyr204), 1:500; pCaMKII(Thr286), 1:1000 (Cell Signaling Technology); GAPDH, 1:1000 (Sigma); GFAP and pCREB (Ser133)(SCT). The filters were then developed using ECL reagents (Perkinelmer) with secondary antibodies (Chemicon). Data was analyzed with a Molecular Imager (Gel Doc™ XR, 170-8170) and the associated software Quantity One-4.6.5 (Bio-Rad Laboratories).

### Immunohistochemistry

Sections of the spinal cord or DRG tissues were incubated with polyclonal rabbit antibodies of anti-EphB1 (1:20) and anti-ephrinB2 (1:20)(SCT) and mouse monoclonal anti-neuronal nuclear protein (NeuN)(1:100)(Alexa Fluor-488 conjugated, MAB377X)(Chemicon). Rabbit IgG (1:200)(Vector Laboratories) was used as an isotype control. Morphologic details were examined with a confocal microscope (Leica TCS SP2, Germany). The protocols were similar to those described previously (Liu WT, et al., FASEB J 2009; 23:90-8; Liu WT, et al., J Neurosci 2010; 30:7613-23).

Sections of the spinal cord or DRG tissues were incubated with polyclonal antibodies of anti-EphB1(1:20), anti-ephrinB2(1:20), rabbit anti-c-Fos(1:100, sc-52, Santa Cruz), rabbit anti-CGRP(1:1000, Millipore), mouse monoclonal anti-neuronal nuclear protein (NeuN) (1:100)(Alexa Fluor-488 conjugated, MAB377X, Chemicon), rabbit polyclonal anti-GFAP (1:500, ab7260, Abeam), anti-IBA (1:100, 019-19741, Wako Pure Chemical Industries), and anti-IB4 (1:100, FITC conjugated) (Sigma), respectively. Rabbit IgG (1:200, Vector Laboratories) was used as an isotype control. Morphologic details of the double immunofluorescent staining were examined with a confocal microscope (Leica TCS SP2, Germany). The number of Fos-like immunoreactive neurons in laminae I-VII was determined by averaging the counts made in 20 spinal cord sections for each group. To obtain quantitative measurements of GFAP and IBA immunofluorescence, 15-20 fields covering the entire DH in each group were evaluated and photographed at the same exposure time to generate the raw data. The average green fluorescence intensity of each pixel was normalized to the background intensity in the same image. These protocols were similar to those described previously (Liu WT, et al., FASEB J 2009; 23:90-8; Liu WT, et al., J Neurosci 2010; 30:7613-23).

### Morphine Tolerance Tests

Each of CD-1, EphB1^{+/+} or EphB1^{-/-} mice was placed on a 55° C. hot plate apparatus and the latency to lick a paw was measured following subcutaneous morphine injection. Protocol for testing acute tolerance (Mor100-10): the latency to lick a paw was measured at 0.5, 1, 1.5, and 2 h following the second dose (10 mg/kg, i.p.) administrated 24 h after the first dose of morphine (100 mg/kg). Protocol for testing chronic tolerance (Mor10): morphine (10 mg/kg) was injected daily for 7 days, and the analgesic effect was measured 30 min after each injection. To examine the role of EphB receptor in morphine tolerance, EphB2-Fc (2 μg, i.t.) or its Fc control was administrated 30 min prior to the morphine at 100 mg/kg in acute tolerance or each of the morphine injection at 10 mg/kg in the chronic tolerance in CD-1 mice.

### Gelatin Zymography

The protocol was similar to that previously described (Liu WT, et al., FASEB J 2009; 23:90-8; Liu WT, et al., J Neurosci 2010; 30:7613-23). The animals were anesthetized deeply and transcardially perfused with PBS and then a length of spinal cord containing segments L₁ to L₆ was rapidly dissected and homogenized in a lysis buffer containing proteinase inhibitors. Aliquots (10 μl) of the homogenates were saved for total protein measurement (BCA kit). The homogenates were centrifuged at 12,000 RPM for 5 min. The supernatants were recovered and incubated for 60 min with gelatin-Sepharose 4B (Pharmacia Biotech) with constant shaking. After incubation, the samples were centrifuged at 500 RPM for 2 min. The pellets were resuspended in 80 μl of elution buffer for 30 min. The entire sample was loaded onto a 8% SDS gel containing 1 mg/ml gelatin. The gel was washed for 3-4 h to remove SDS and allow renaturation of MMPs. The gel was then left for 48 h in an incubation buffer to allow MMPs to degrade the gelatin in their immediate vicinity. Finally, zones of gelatin degradation representing proteolytic activity were identified by staining the gel with Coomassie blue and de-staining with methanol and acetic acid in water (3:1:6).

### X-Ray and Histological Observations

To verify bone destruction and tumor cell infiltration, X-ray and histological observations of the affected bone were made after TCI. The X-ray images were taken on postoperative 14 and 21 days (exposure setting: 12 ms, 31 KVp) using Philips Digital Radiographer System (Digital diagnost VM, Philips Medical Systems DMC GmbH, Hamburg, Germany). Histological observations of the affected bone were made on the 14^{th} day after TCI. Rats were anesthetized and transcardially perfused with 0.9% saline (150 ml) followed with 4% paraformaldehyde (300 ml). The tibia bones were removed and demineralized in EDTA (10%) for 2 weeks. Then the tibiae were embedded in paraffin, and 5 μm sections were cut with a microtome and stained with hematoxylin and eosin (HE) for analysis under a microscope.

### Statistical Analyses

SPSS Rel 15 was used to conduct all the statistical analyses. Alteration of expression of the proteins detected and the behavioral responses to thermal and mechanical stimuli over time among groups were tested with one-way and two-way ANOVA with repeated measures followed by Bonferroni post hoc tests, respectively. All data are presented as means±SEM. Statistical results are considered significant if p<0.05.

### Results

### Activity and Distribution of EphB1 and EphrinB2 in Spinal Cord Following TCI

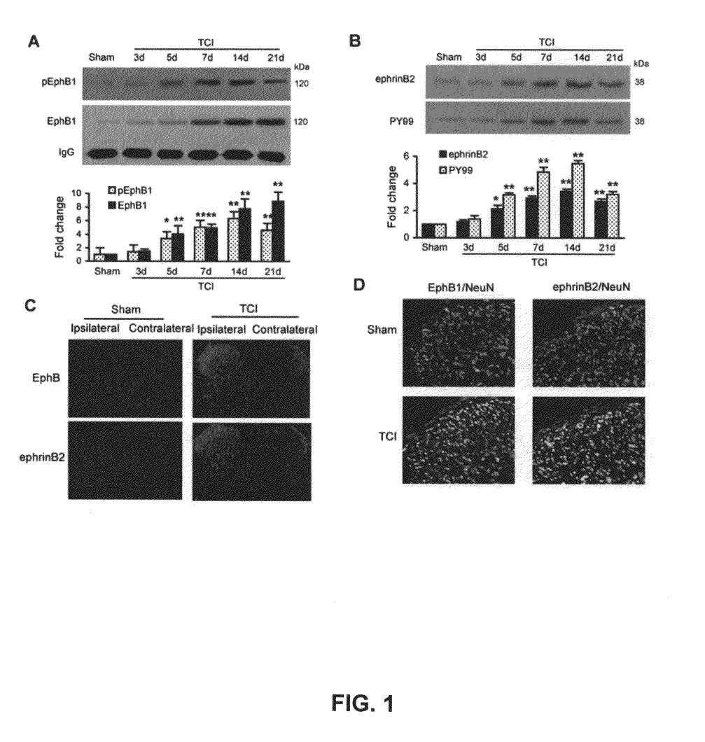

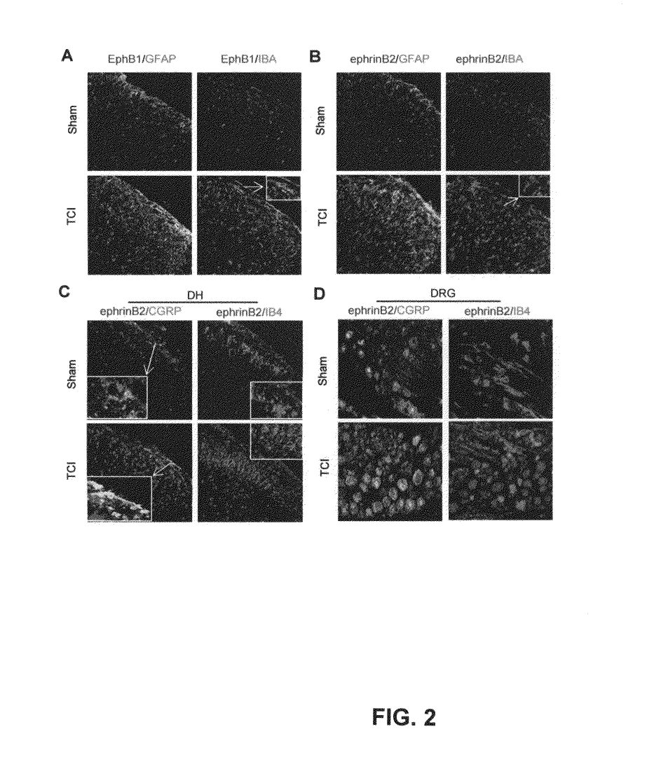

Level of protein expression and phosphorylation of EphB1 receptor and its ligand ephrinB2 in the spinal cord was increased significantly in a time-dependent manner after TCI, while they were at low levels in naïve rats (FIGS. 1A-1B). Phosphorylation of EphB1 was slightly decreased on the postoperative 21 days, but not altered during postoperative 3-14 days since ratio of EphB1 and pEphB1 was remained the same. The increased EphB1 and ephrinB2 immunoreactivity were distributed predominately in the superficial dorsal horn (DH) ipsilateral to TCI (FIG. 1C). EphB1 immunoreactivity was colocalized primarily with neurons (FIG. 1D) and astrocytes, while only a small amount with microglia (FIG. 2A). EphrinB2 immunoreactivity was colocalized primarily with neurons (FIG. 1D) and astrocytes, but not with microglia (FIG. 2B). In Primary afferent terminals within the DH and the nociceptors within the DRG, ephrinB2 immunoreactivity was colocalized with peptidergic CGRP-, but not the non-peptidergic IB4-fibers and cells (FIGS. 2C-2D). These results demonstrate that TCI can cause an upregulation of EphB1 and ephrinB2 expression in the neurons and astrocytes within the DH; the ephrinB2 may originate, at least partly, from the peptidergic CGRP-nociceptive neurons.

### Blocking EphB1 Receptor Activation Prevents and Suppresses Behavioral and Neurochemical Signs of Bone Cancer Pain

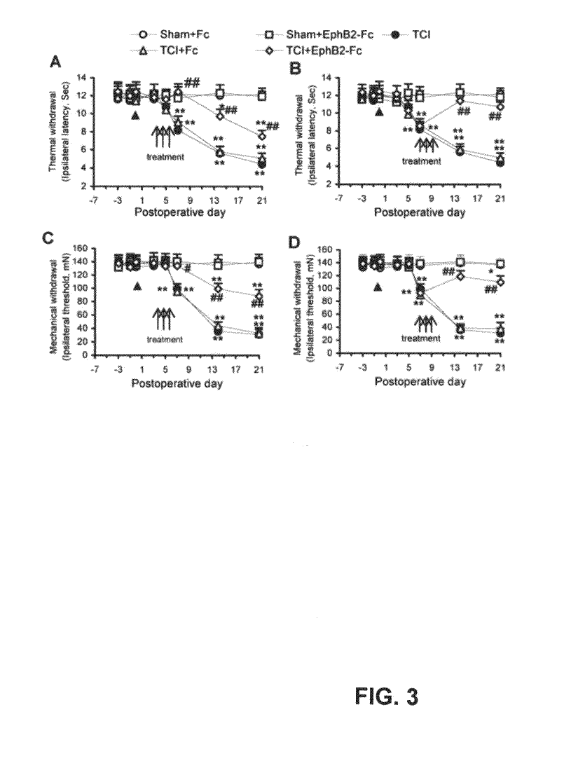

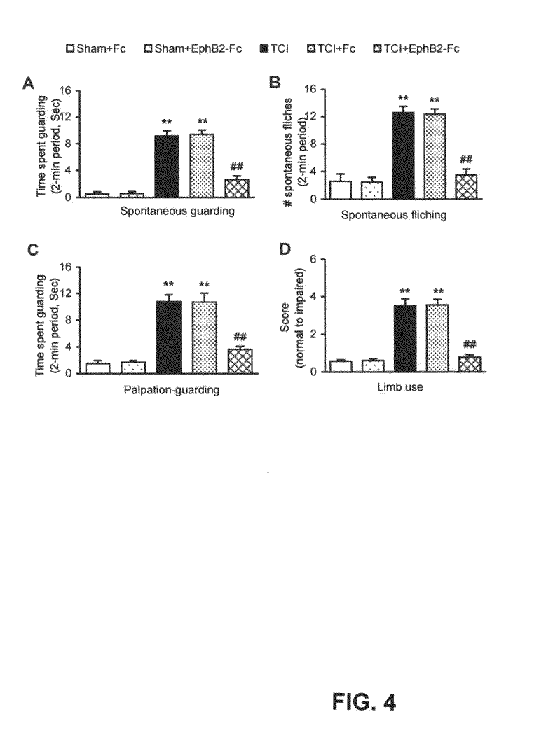

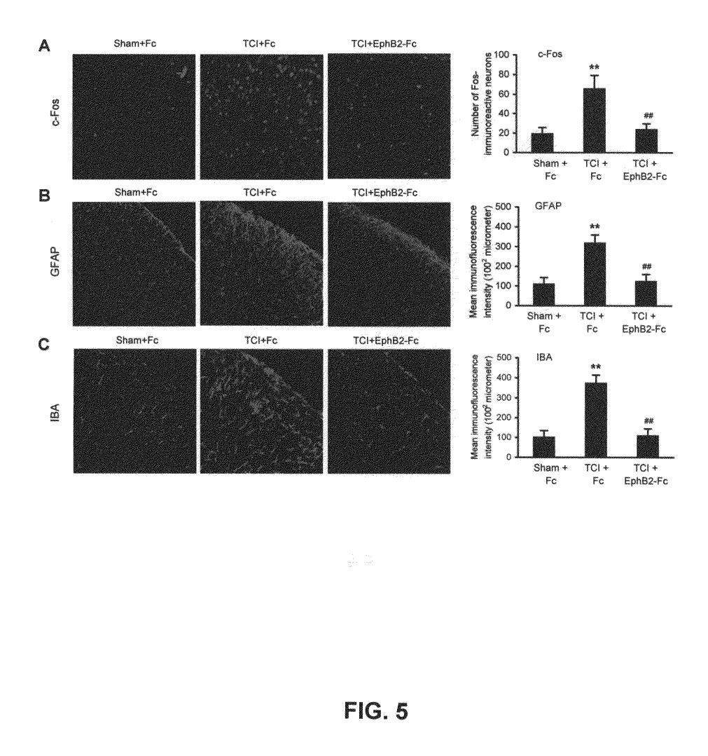

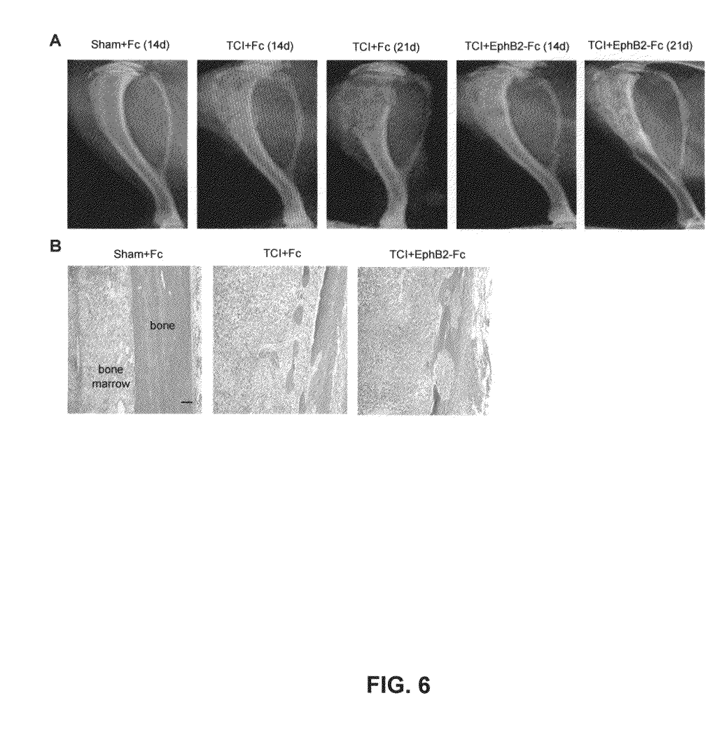

Rats that received TCI exhibited thermal hyperalgesia and mechanical allodynia (FIG. 3). TCI rats also exhibited spontaneous pain manifested as guarding and movement-evoked pain manifested as guarding, flinching and reduced limb use (FIG. 4). Production and persistence of these pain-related behaviors were greatly delayed and suppressed by an EphB1 receptor inhibitor EphB2-Fc administrated (each 5 μg, i.t., once a day) at postoperative 3, 4 and 5 day (FIGS. 3A and 3C) and postoperative 7, 8 and 9 day (FIGS. 3B and 3D and FIG. 4), respectively. Normal pain sensation was not altered. In addition, TCI caused induction of c-Fos and activation of astrocytes and microglial cell in the DH ipsilateral to TCI. These alterations were inhibited by repetitive treatments with EphB2-Fc (FIG. 5). These results suggest that ephrinB-EphB receptor signaling may play an important role in production and persistence of bone cancer pain. In addition, pathological observations (X-ray and hematoxylin-eosin staining) showed that bone destruction associated with tumor growth seemed to be slightly improved following such transient EphB2-Fc treatment (FIG. 6).

Blocking EphB1 Receptor Activation Suppresses TCI-Induced Increase of Level of pNR1, pNR2B, pSrc, pERK, pCaMKII and pCREB in Spinal Cord

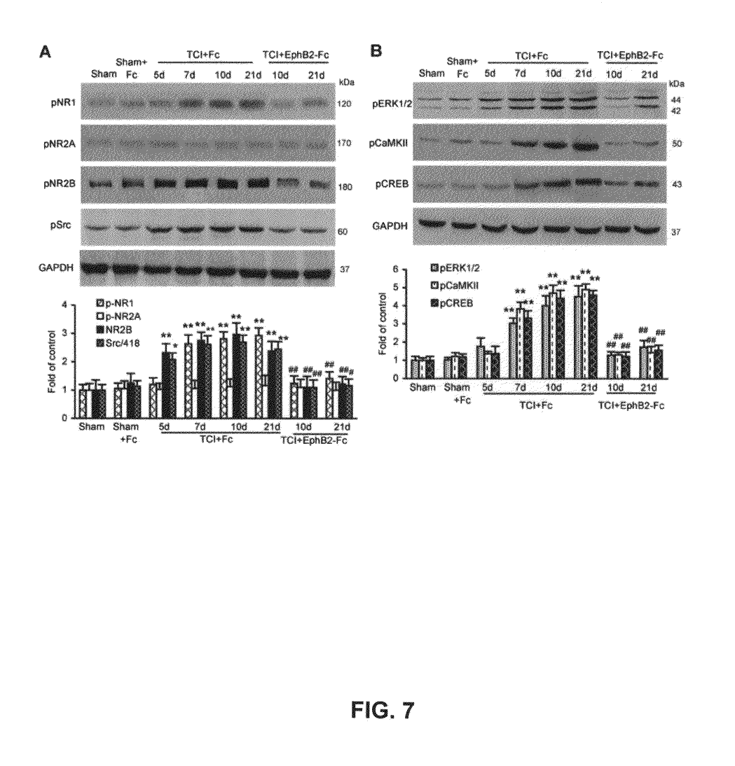

NMDARs have a well-developed role in neural plasticity and in various pain states. Src-family kinases can enhance NMDAR function (Yu X M, et al., Proc Natl Acad Sci USA 1999; 96:7697-704; Salter M W, et al., Nat Rev Neurosci 2004; 5:317-28; and Liu Xi, et al., Nat Med 2008; 14:1325-32) and ephrinB2 may activate NR2B receptor via Src during inflammatory hyperalgesia (Slack S, et al., Neuroscience 2008; 156:175-83). TCI treatment significantly increased levels of pNR1, pNR2B, pSrc (Tyr418) (FIG. 7A), pERK, pCaMKII and pCREB (FIG. 7B). Timing patterns of upregulation of these phosphorylated molecules were well matched with that of bone cancer pain-related behaviors (FIG. 3). Repetitive treatment with EphB2-Fc (each 5 once a day on postoperative 7, 8 and 9 day, respectively) inhibited the expression of these molecules. (FIG. 7). Expression of pNR2A was neither altered by TCI nor EphB2-Fc treatment (FIG. 7A). These results suggest that TCI-induced activation of NR1 and NR2B receptors, Src and the subsequent Ca²⁺-dependent signals may be secondary to activation of the ephrinB-EphB receptor signaling.

### EphB2-Fc Inhibits TCI-Induced Increase in Expression of EphB1, but not EphrinB2

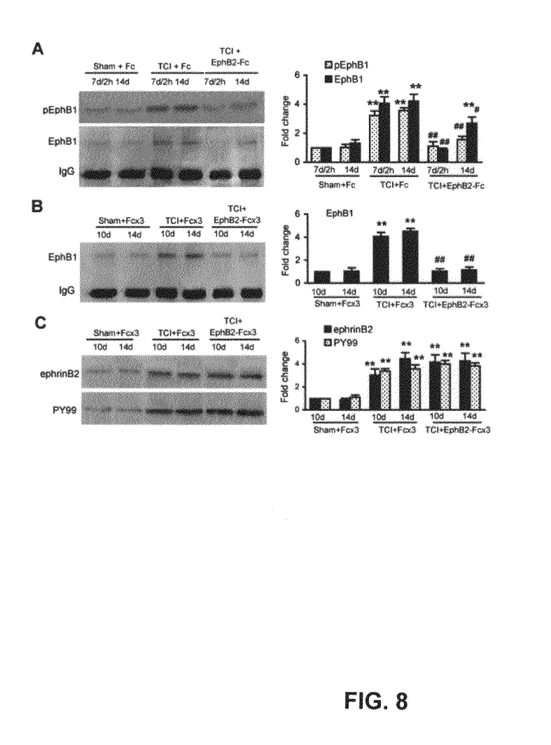

EphrinB-EphB interaction is well known to lead bidirectional signals, the forward- and the reverse-signaling (Klein R. et al., Nat Neurosci 2009; 12:15-20). There is no evidence if either the forward- or the reverse-signaling or both is necessary for production and maintenance of any pain status. The instant study showed that TCI-induced upregulation of expression of EphB1 receptor was cancelled by EphB2-Fc (5 μg, i.t.) in a single dose on the postoperative 7 day (FIG. 8A) or repetitive doses on postoperative 7, 8 and 9 day, respectively (FIG. 8B). Inhibition occurred within 2 h and persisted for at least a week. EphB2-Fc treatment did not affect TCI-induced increase of expression of ephrinB2 and PY99 (FIG. 8C). These results, together with those shown in FIG. 7, indicate that EphB1 downregulation may contribute to inhibition of pNR1, pNR2B, pSrc(Tyr418), pERK1/2, pCaMKII and pCREB as well as reduction of bone cancer pain behaviors following EphB2-Fc treatment; while continuous activation of the ephrinB2 is unable to maintain activation of these signals and the pain status.

EphrinB2-Fc Activates NR1 and NR2B and Induces Thermal Hyperalgesia on Condition of EphB1 Receptor Activation in Naïve Rats

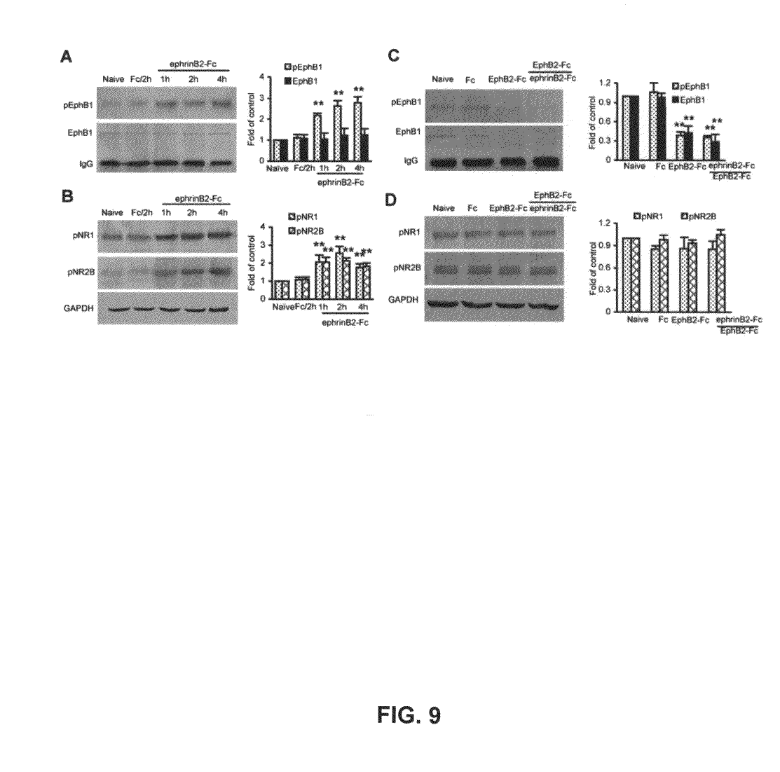

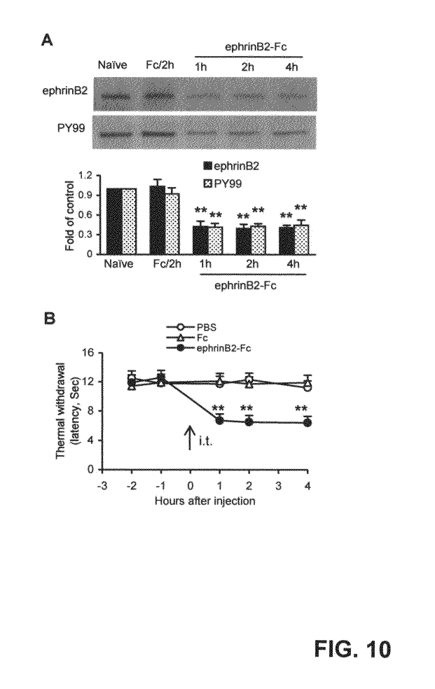

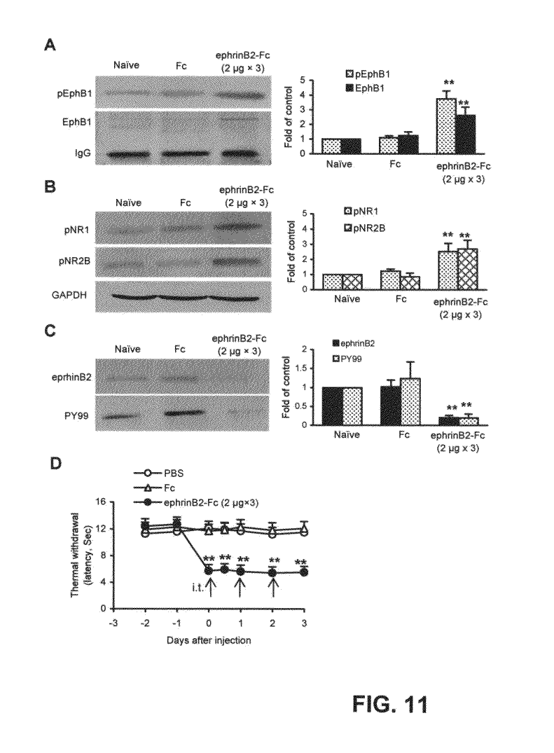

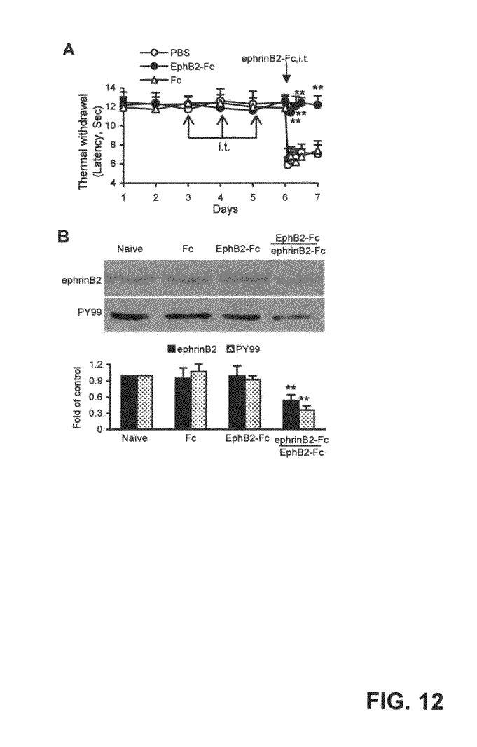

Spinal administration of an exogenous EphB1 receptor activator ephrinB2-Fc (2 μg) increased the level of pEphB1, but not the overall receptor expression of EphB1 (FIG. 9A), and upregulated levels of pNR1 and pNR2B (FIG. 9B). It also decreased ephrinB2 expression, but not its phosphorylation PY99, since the ratio ephrinB2/PY99 was maintained at the same level (FIG. 10A), and resulted in thermal hypersensitivity (FIG. 10B). Repetitive ephrinB2-Fc (2 μg, i.t., once a day for 3 consecutive days) produced similar effects on phosphorylation or expression of these molecules and thermal hypersensitivity (FIG. 11). It was further examined if ephrinB2-Fc-induced activation of NR1 and NR2B and thermal hypersensitivity would be dependent on activation of the EphB1 receptor. TCI-induced upregulation of EphB1 was inhibited by EphB2-Fc treatment (FIG. 8). In naïve rats, EphB2-Fc treatment (5 μg, for 3 consecutive days) greatly inhibited expression of EphB1 and its phosphorylation (FIG. 9C). Under this condition, ephrinB2-Fc (2 μg) failed to activate or rescue EphB1 (FIG. 9C), NR1 and NR2B (FIG. 9D), but continued to down-regulate ephrinB2 (FIG. 12A). Additionally, ephrinB2-Fc treatment caused thermal hypersensitivity in naïve rats, but failed to do so when co-administrated with EphB2-Fc (FIG. 12B). These results indicate that activation of EphB1 receptor is required for ephrinB2-Fc to activate NR1 and NR2B and further induce thermal hypersensitivity, suggesting that EphB1 and NR1/2 activation is necessary for the development of thermal hyperalgesia. Downregulation of ephrinB2 is not sufficient or unnecessary for ephrinB2-Fc-induced thermal hypersensitivity.

Inhibition of MMP-2 or MMP-9 Activation Prevents EphB2-Fc-Induced Down-Regulation of EphB1 in Naïve and TCI Rats

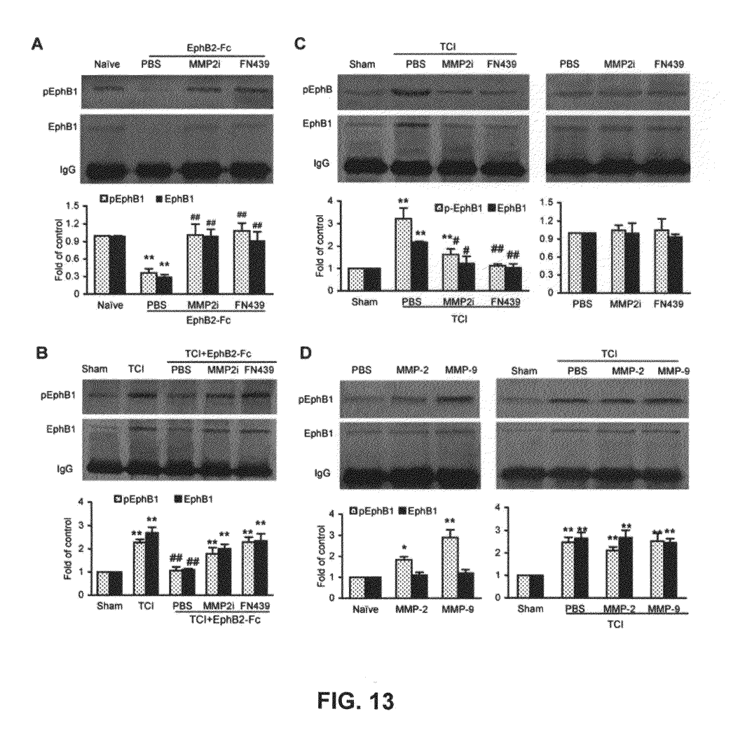

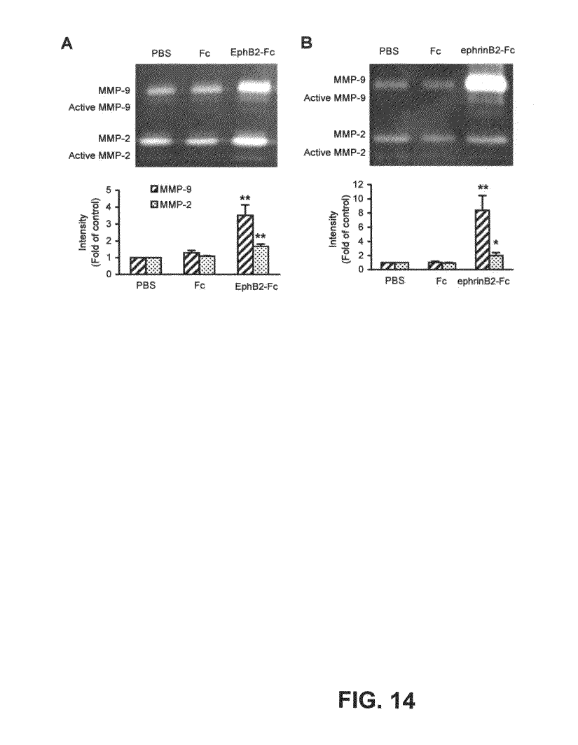

Spinal administration of EphB2-Fc results in downregulation of EphB1 protein, in addition to inhibition of TCI-induced pain-like behaviors and neurochemical alterations. Since EphB-ephrinB interactions can be broken by MMPs, which cleaves EphB receptors (Hattori M, et al., Science 2000; 289:1360-5; Janes P W, et al., Cell 2005; 123:291-304; and Lin K T, et al., J Biol Chem 2008; 283:28969-79), possible roles of spinal MMP-9/2 in EphB2-Fc-induced EphB1 down-regulation were examined. With EphB2-Fc treatment, levels of EphB1 and pEphB1 were further downregulated from already low levels in naïve rats (FIG. 13A) and, in TCI rats, back to control (Sham) levels from greatly increased levels (FIG. 13B), respectively. The EphB2-Fc-induced downregulation of EphB1 was completely prevented by blocking MMP-2 with MMP2i (10 μg) or MMP-9 with FN439 (100 μg) (FIGS. 13A-13B) and the level of EphB1 was brought back to levels originally seen in naïve (FIG. 13A) and TCI (FIG. 13B) rats, respectively. However, without EphB2-Fc treatment, blocking MMP-2 or MMP-9 inhibited TCI-induced upregulation of EphB1 and brought the upregulated EphB1 back to control (Sham) levels (FIG. 13C, left), while keeping EphB1 at its physiological levels in naïve rats (FIG. 13C, right). Administration of exogenous MMP-2 or MMP-9 caused an increase rather than a decline in the activation (phosphorylation) of EphB1 without altering the expression of EphB1 protein in naïve rats (FIG. 13D, left) and did not significantly alter increased levels of EphB1 in TCI rats (FIG. 13D, right). Gelatin zymography showed that activity of both MMP-2 and MMP-2 was greatly increased following EphB2-Fc or ephrinB2-Fc treatment (FIG. 14). These results indicate that MMP-2/9 have different roles, with and without EphB2-Fc treatment, in the modulation of EphB1. With EphB2-Fc treatment, they held EphB1 back to the reference levels which are relatively low in naïve rats and greatly increased in TCI rats. Without EphB2-Fc treatment, they may upregulate the EphB1 level.

### Blocking EphB1 Receptor Activation Rescues Analgesic Effect of Morphine in Treating Bone Cancer Pain

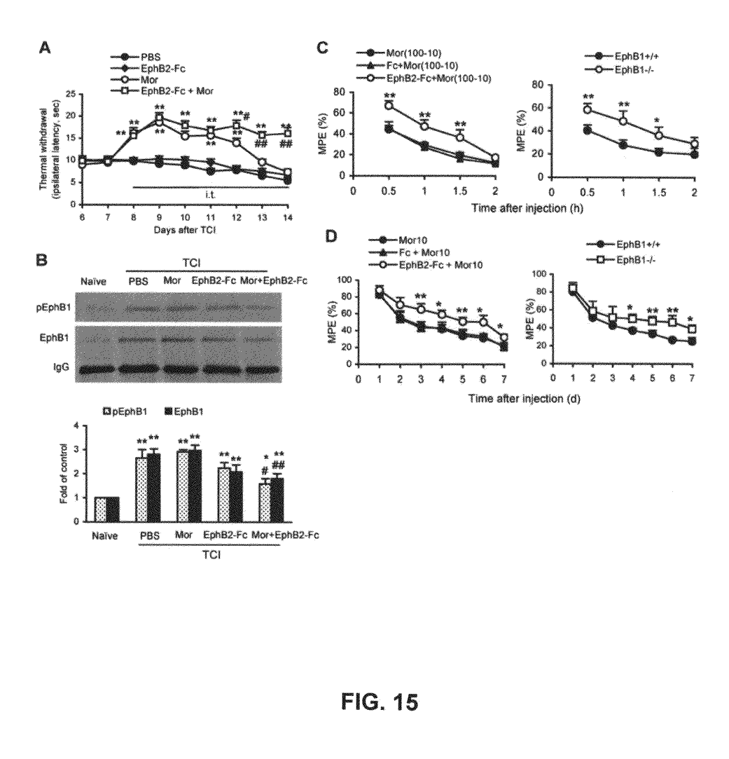

Morphine has been used in reducing cancer pain, particularly in advanced and terminal patients, but its tolerance following repeated use has limited its clinical use. Given that EphB1 receptor may be a potential target for treating bone cancer pain, the experiments were extended to examine whether morphine tolerance in treating bone cancer pain would be involved in the activation of ephrinB-EphB signaling. Injection of morphine (10 mg/kg, s.c., twice a day, 12 h intervals) significantly suppressed thermal hyperalgesia in TCI rats. Such an analgesic effect decreased and then disappeared (tolerance) 6-7 days after repeated use of morphine (FIG. 15A). Co-administration of EphB2-Fc at lower doses (each 2 μg accompanied with morphine), which alone was not enough to reduce the hyperalgesia, successfully rescued the analgesic effect of morphine (prevented morphine tolerance) in treating bone cancer hyperalgesia (FIG. 15A). Meanwhile, increased level of phosphorylation and expression of EphB1 following TCI and morphine treatment were inhibited by repetitive EphB2-Fc treatment (FIG. 15B).

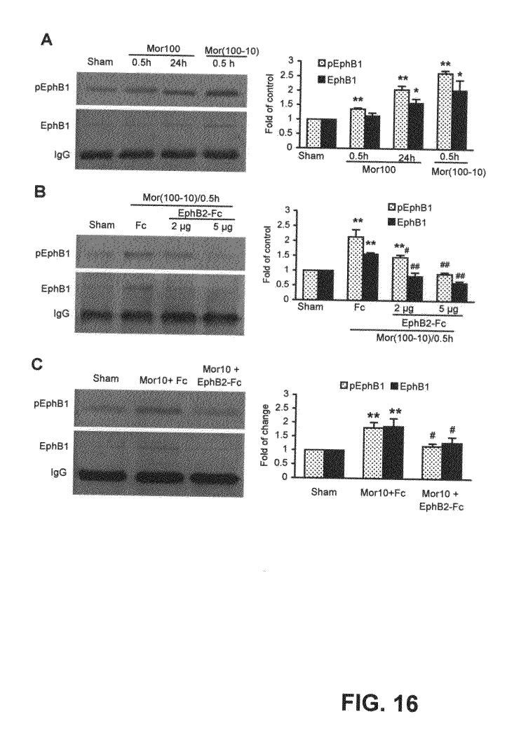

The role of EphB1 receptor in morphine tolerance was further investigated using standard protocols for testing acute and chronic morphine tolerance in naïve and EphB1 receptor knock out (EphB1^{-/-}) mice. Both acute (FIG. 15C) and chronic (FIG. 15D) morphine tolerance were reduced by co-administration of EphB2-Fc (each 2 μg, i.t.) with morphine or by targeted mutation of EphB1. Meanwhile, acute (FIGS. 16A-16B) and chronic (FIG. 16C) morphine treatment significantly increased expression of EphB1 and its phosphorylation, and this effect was inhibited by EphB2-Fc (FIGS. 16B-16C). These results indicate that the EphB1 receptor is involved in development of morphine tolerance.

### Discussion

The instant study reveals a critical role for the ephrinB-EphB receptor signaling in cancer pain, for example, bone cancer pain, and morphine tolerance in treating the cancer pain. TCI greatly activates spinal ephrinB2-EphB1 receptor signaling, which then induces activation of NR1 and NR2B receptors and the downstream pathways, resulting in the cancer pain, for example, bone cancer pain. EphB1 is required for the production and persistence of the cancer pain and the functional link between the ephrinB-EphB receptor signaling, NR1 and NR2B receptors and downstream responses. Cleavage of EphB1 receptor may be mediated by MMP-2 and MMP-9. These findings provide a novel mechanism underlying the pathogenesis of the cancer pain, for example, bone cancer pain. EphB1 receptor is also required for the development of morphine tolerance. Thus, EphB1 receptor may be a potential target for treating cancer pain and reducing opioid tolerance in treating cancer pain clinically. A representative example of cancer pain is bone cancer pain.

EphB receptors and their ligands, ephrinBs, are critical cues in regulating cell shape, adhesion/repulsion, migration and positioning during developmental processes (Wang H U, Anderson D J., Neuron 1997; 18:383-96; Wilkinson D G., Int Rev Cytol 2000; 196:177-244; Pasquale E B., Nat Rev Mol Cell Biol 2005; 6:462-75; and Krull C E, et al., Curr Biol 1997; 7:571-80). Alteration of mechanisms controlling adhesion and motility has a central role in promoting tumor invasion and angiogenesis (Friedl P, et al., Nat Rev Cancer 2003; 3:362-74; Christofori G., Nature 2006; 441:444-50; and Sahai E., Curr Opin Genet Dev 2005; 15:87-96). The instant study shows that TCI-induced activation of EphB1 receptor and its ligand ephrinB2 are well correlated with the timing -patterns of TCI-induced bone cancer pain behaviors. The increased EphB1 receptor and ephrinB2 are predominantly localized within neurons and some astrocytes in the DH, an essential area for the generation and processing of pain signals. EphrinB2 in the peripheral nociceptors may be transported and redistributed along the CGRP-fibers, to the pre-synaptic membranes of their central terminals, where it triggers a series of postsynaptic effects. EphB receptors regulate synaptic plasticity by interaction with NMDARs (Dalva M B, et al., Cell 2000; 103:945-56; Nestler E J., Nat Rev Neurosci 2001; 2:119-28; and Takasu M A, et al., Science 2002; 295:491-5). NMDARs have a well-developed role in neural plasticity and in various pain states. Within the NMDAR complex, the non-receptor tyrosine kinase, Src, provides dynamic-gain-control of NMDAR function via balancing its activity with striatal enriched tyrosine (Yu X M, et al., Proc Natl Acad Sci USA 1999; 96:7697-704; Salter MW, et al., Nat Rev Neurosci 2004; 5:317-28; Liu X J, et al., Nat Med 2008; 14:1325-32; Slack S, et al., Neuroscience 2008; 156:175-83; and Pelkey K A, et al., Neuron 2002; 34:127-38). It is also shown in the instant study that blocking EphB1 receptor activation largely diminished TCI-induced phosphorylation of NR1 and NR2B receptors, Src(Tyr418), and subsequent activation of various Ca²⁺-dependent signaling enzymes, CREB, induction of c-Fos and bone cancer-related pain behaviors. Therefore, it is hypothesized that peripheral nerve damage due to cancer cell invasion leads to upregulation of the presynaptic ephrinB2 ligand and postsynaptic EphB1 receptor molecules in primary neurons and the DH. Such an increased expression of ephrinB-EphB signaling then leads to postsynaptic EphB1 receptor forward signaling clustering and reciprocal phosphorylation on multiple tyrosine residues (Dalva MB, et al., Cell 2000; 103:945-56; Grunwald I C, et al., Nat Neurosci 2004; 7:33-40). Thus, downstream signaling proteins containing SH2 domains, including Src family kinases (Grunwald IC, et al., Nat Neurosci 2004; 7:33-40; Murai K K, et al., Neuroscientist 2004; 10:304-14), which are physically associated with EphB receptors and NMDARs and phosphorylate NMDARs on tyrosines (Nakazawa T, et al., J Biol Chem 2001; 276:693-9) may be recruited. This results in the subsequent activation of Ca²⁺-dependent signaling pathways and behavioral manifestations.

It is noted that ephrinB2-Fc treatment and TCI induced thermal hyperalgesia may involve EphB1 receptor, probably via two different mechanisms. EphrinB2-Fc treatment may induce true EphB1 receptor activation, since it does not affect overall receptor expression, but increases the level of phosphorylation of EphB1. EphrinB2-Fc treatment also down-regulates expression of both ephrinB2 and PY99. On the contrary, TCI induces an upregulation of both EphB1 and pEphB1, and ephrinB2 and PY99.

EphrinB-EphB interaction is well known to lead bidirectional signals which are propagated into the ephrinB-expressing cells (reverse signaling) and the EphB-expressing cells (forward signaling). It is unknown if either of the forward- or reverse-signaling or both are important in pain processing. The instant findings in this bone cancer model may support that EphB1 receptor forward signaling is critical to the development and maintenance of pain and activation of NR1 and NR2B receptors as well as subsequent Ca²⁺-dependent signals. When EphB1 is inhibited, pain behaviors and NMDARs activation are inhibited even though the ephrinB2 continues to be active. When EphB1 receptor is activated, NMDARs are activated and pain is induced even though ephrinB2 is down-regulated. A recent study, by deleting ephrinB2 in Nav1.8⁺ nociceptive sensory neurons, indicates that presynaptic ephrinB2 expression may play a role in regulating inflammatory pain and some types of neuropathic pain through the regulation of synaptic plasticity in the dorsal horn (Zhao J, et al., Mol Pain 2010; 6:77).

It is also noted that EphB2-Fc treatment results in downregulation of EphB1 protein accompanied with increased activity of MMP-2 and MMP-9 in the spinal cord. Such EphB1 downregulation can be prevented or rescued by blocking MMP-2 or MMP-9. It is hypothesized that, during EphB2-Fc treatment, EphB2-Fc combines with endogenous ephrinB2 and thus EphB1 is substituted and cleaved (downregulation) mediated by MMP-2 and/or MMP-9. This results in inhibition of the downstream signals of the EphB1. In contrast, without EphB2-Fc treatment, exogenous ephrinB2-Fc or TCI-induced increased ephrinB2 combines with EphB1 and results in upregulation of EphB1 and activation of the downstream signals. In this process, neither ephrinB2-Fc nor ephrinB2 causes downregulation of EphB1 although it induces activation of MMP-2 and MMP-9, thus, blocking MMP-2 or MMP-9 does not cause downregulation of EphB1. In summary, MMP-2/9 may mediate EphB1 cleavage during EphB-Fc treatment and thus enhance the inhibitory effect of EphB2-Fc on EphB1 signaling; while MMP-2/9 alone may activate rather than cleave EphB1.

Cancer pain posses a major challenge clinically. Opioid drugs, with or without coanalgesics/adjuvant analgesics, are used to treat moderate to severe pain. However, the effective use of morphine as a treatment on such pain is limited by morphine tolerance and concerns related to addiction and opioid administration-associated severe, sometimes debilitating side effects (Blum R H, et al., Oncology (Williston Park) 2003; 17:845-57; discussion 62-3, 67; Mercadante S, et al., J Pain Symptom Manage 2003; 26:691-3; Silverman S M. Pain Physician 2009; 12:679-84; and Guan J S, et al., Cell 2005; 122:619-31). The instant study demonstrates that pharmacological blockade or genetic deletion of EphB1 receptor can prevent morphine tolerance in treating bone cancer pain and defensive pain, respectively. In addition, it has recently been found that inhibition of EphB1 receptor can prevent or largely diminish morphine physical dependence (Liu WT, et al., FASEB J 2009; 23:90-8). Thus, blocking EphB1 receptor may be a potential pharmaceutical therapy that can not only relieve cancer pain, but also facilitate the clinical utility of opioid drugs in treating cancer pain. A representative example of cancer pain is bone cancer pain.

While the invention has been shown in only a few of its forms, it should be apparent to those skilled in the art that it is not so limited but susceptible to various changes without departing from the scope of the invention.

What is claimed is:

1. A method for treating cancer pain, comprising:
administering to an individual in need of such treatment with a pharmaceutically effective amount of a blocking reagent for ephrinB-EphB signaling.

2. The method of claim 1, wherein said blocking reagent is an EphB receptor blocker.

3. The method of claim 2, wherein said EphB receptor blocker comprises EphB1-Fc.

4. The method of claim 2, wherein said EphB receptor blocker comprises EphB2-Fc.

5. The method of any one of claims 1 to 4, wherein said blocking reagent is administered in an amount of from about 0.5 μg to about 75 mg.

6. The method of claim 5, wherein said blocking reagent is administered in an amount of from about 1 μg to about 15 mg.

7. The method of claim 1, wherein said blocking reagent is administrated intrathecally.

8. The method of claim 1, wherein said cancer is a primary bone sarcoma and secondary bone cancer, wherein said secondary bone cancer occurs as a distant metastasis of a non-bone primary tumor in breast, prostate or lung of said individual.

9. The method of claim 1, wherein said individual is a mammal.

10. The method of claim 9, wherein said mammal is human.

11. A method for reducing opioid tolerance in treating cancer pain thereby rescuing analgesic effect of morphine treatment of cancer pain, comprising:
administering to an individual in need of such treatment with a pharmaceutically effective amount of a blocking reagent for ephrinB-EphB signaling.

12. The method of claim 11, wherein said blocking reagent is an EphB receptor blocker.

13. The method of claim 12, wherein said EphB receptor blocker comprises EphB1-Fc.

14. The method of claim 12, wherein said EphB receptor blocker comprises EphB2-Fc.

15. The method of any one of claims 11 to 14, wherein said blocking reagent is administered in an amount of from about 0.5 n to about 75 mg.

16. The method of claim 15, wherein said blocking reagent is administered in an amount of from about 1 μg to about 15 mg.

17. The method of claim 11, wherein said blocking reagent is administrated intrathecally.

18. The method of claim 11, wherein said cancer is a primary bone sarcoma and secondary bone cancer, wherein said secondary bone cancer occurs as a distant metastasis of a non-bone primary tumor in breast, prostate or lung of said individual.

19. The method of claim 11, wherein said individual is a mammal.

20. The method of claim 19, wherein said mammal is human.
